(19) United States
(12) Patent Application Publication
(10) Pub. No.: US 2012/0065104 A1
(43) Pub. Date: Mar. 15, 2012

(54) COMPOSITIONS AND METHODS FOR A MEMBRANE PROTEIN CRYSTALLIZATION SCREENING KIT

(75) Inventors: Michael C. Wiener, Charlottesville, VA (US); Michael D. Purdy, Staunton, VA (US)

(73) Assignee: UNIVERSITY OF VIRGINIA PATENT FOUNDATION, Charlottesville, VA (US)

(21) Appl. No.: 13/321,716

(22) PCT Filed: May 28, 2010
(86) PCT No.: PCT/US10/36562

§ 371 Date: Nov. 22, 2011

Related U.S. Application Data
(60) Provisional application No. 61/181,746, filed on May 28, 2009.

(51) Int. Cl. C40B 30/10; C07K 14/00; C09K 3/00; C40B 40/00

(52) U.S. Cl. 506/12; 506/13; 530/350; 252/182.12

(57) ABSTRACT

The present invention comprises compositions and methods useful as a system for efficiently determining conditions that result in the formation of crystals of membrane proteins from solutions containing a membrane protein in a purified and soluble state. The system is comprised of two primary components, a solubility screen and a crystallization screen. Each component is a set of solutions. The present invention further provides a kit comprising solutions of the invention and an instructional material for the use thereof.

### CROSS REFERENCE TO RELATED APPLICATIONS

This application claims priority under 35 U.S.C. §119(e) to U.S. Provisional Application Ser. No. 61/181,746 filed May 28, 2009, the disclosure of which is incorporated by reference in its entirety herein.

### STATEMENT REGARDING FEDERALLY SPONSORED RESEARCH OR DEVELOPMENT

This invention was made in part with United States Government support under Grant No. 5R01GM075931, awarded by the National Institutes of Health. The United States Government has certain rights in the invention.

### BACKGROUND

Membrane proteins comprise between 15% and 39% of the human proteome and 45% of drugs target these proteins [2]. Membrane proteins are prevalent in the proteomes of pathogenic microorganisms and are the targets of many antimicrobial agents. Membrane proteins play essential roles in pathophysiology and the biology of all organisms. Near atomic resolution structures are required for our understanding of the function of these molecules. X-ray crystallography, electron crystallography and nuclear magnetic resonance spectroscopy (NMR) are the currently available methods for obtaining high resolution structures of macromolecules, including membrane proteins. Purified membrane proteins require surfactants, typically detergents, to remain soluble in an aqueous environment. The complex of the membrane protein and the associated detergent molecules (the protein detergent complex, PDC), is the object studied by x-ray crystallography or NMR. The ability to solve a structure by NMR is limited by the mass of the molecule. The mass of many PDCs prevents the use of NMR for structure determination, although, the mass limit of NMR is increasing. High resolution electron crystallography of membrane proteins requires formation of a two-dimensional crystal in a lipid bilayer. Very few membrane proteins have yielded two-dimensional crystals of sufficient quality for high resolution structure determination, and the process of structure determination by electron crystallography is arduous. X-ray crystallography is the method used most commonly to solve structures of membrane proteins. However, the number of solved membrane protein structures lags far behind that of soluble proteins. Crystallization is a primary obstacle to solving the structures of membrane proteins by x-ray crystallography.

Crystallization of proteins, including membrane proteins, typically involves mixing the purified protein with solutions intended to drive the protein to supersaturation, crystal nucleation, and crystal growth [3]. Usually hundreds or thousands of crystallization solutions are tried with each new protein. A large number of protein crystallization screens, sets of crystallization solutions, are available commercially, including several specifically designed for membrane protein crystallization.

There is a long felt need in the art for compositions and methods useful as a system for efficiently determining conditions that result in the formation of crystals of membrane proteins from solutions containing a membrane protein in a purified and soluble state. The present invention satisfies these needs.

### SUMMARY OF THE INVENTION

The present invention is designed to address the problem of protein crystallization, particularly membrane protein crystallization. The present invention improves on the prior art in two ways. The system includes a solubility pre-screen which is used to determine the proper membrane protein concentration for use with the crystallization screen, which is then followed by use with the crystallization screen solutions of the invention. The present invention provides compositions and methods useful to reduce the number of crystallization experiments necessary to obtain crystals of membrane proteins suitable for x-ray diffraction experiments and structure determinations. Therefore, the present invention provides compositions and methods for first determining the solubility of a protein, which then allows a more optimal protein concentration to be used in the protein crystallization screening method of the invention.

In one embodiment, the present invention is a system for efficiently determining conditions that result in the formation of crystals of proteins from solutions containing a protein in a purified and soluble state. In one aspect, the protein is a membrane protein.

In one embodiment, the present invention provides compositions and methods for a solubility screen, as well as a kit. In one aspect, the solubility screen comprises from one to twenty-four distinct formulation solutions, each of which comprises a precipitant, optionally a buffer, and optionally a salt. In one aspect, all twenty-four formulations are used in the solubility screening process. By “prescreening the solubility of a protein” is meant that the solubility screens described herein are used to determine better or optimum conditions for a crystallization screen. The intended purpose of the solubility screen is to determine the concentration of a membrane protein that renders a crystallization screen more useful. Specifically, the concentration of a membrane protein must be such that the majority of the crystallization screen solutions result in supersaturation of the protein without excessive non-crystalline precipitation.

In one embodiment of the invention, two samples of a membrane protein are prepared, one sample near the solubility limit of the membrane protein, and another sample of the membrane protein at half that concentration. The membrane protein, at each concentration is then mixed with the solutions of the solubility screen at a 1:1 volumetric ratio. The mixtures are then inspected using an optical microscope for the presence of protein precipitate. The solubility screen solutions correspond to the low and high extrema of the precipitant concentrations in the crystallization screen. The optimal concentration of the membrane protein, for use in the crystallization screen, is indicated by clear solutions for low precipitant concentrations and solutions containing protein precipitate for high precipitant concentrations.

Formulations of the solubility screen of the invention include, but are not limited to, those exemplified in Table 2 and numbered 1-24.

TABLE 2
Solubility Screen Solution Formulations.

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1 |

In one embodiment, the crystallization screen of the invention comprises ninety-six distinct formulation solutions each of which comprises a precipitant, and optionally a buffer, optionally a salt, and optionally up to two additives. A solution comprising a purified membrane protein is mixed with each of the crystallization formulation solutions. In one aspect, the mixing is performed in a vapor diffusion crystallization experiment. The precipitants and salt additives are those which were used most frequently in successful membrane protein crystallization experiments at the time of the development of the screen (data from, the Membrane Protein Data Bank “MPDB”, available at a website maintained at the University of Dublin Trinity College). In each crystallization solution, the precipitant is a polyethylene glycol (PEG) or a polyethylene glycol monomethylether (MPEG). The role of the PEG or MPEG is to reduce the solubility of the protein detergent complex (PDC) and stimulate crystal nucleation [1].

In one embodiment, one of six buffers or no buffer is included in each crystallization solution. The pH of the buffers varies from pH 4.5 to pH 9.5. The role of the buffers is to alter the surface properties of the membrane proteins. Useful buffers of the invention include, but are not limited to, sodium citrate, sodium acetate, ADA, HEPES, Tris HCl, and CAPSO.

In one aspect, one of eighteen salts or no additional salt is included in each condition of the crystallization screen. Useful salts of the invention include, but are not limited to, ammonium nitrate, ammonium sulfate, calcium chloride, lithium chloride, lithium nitrate, lithium sulfate, magnesium acetate, magnesium chloride, magnesium sulfate, potassium chloride, potassium phosphate dibasic, potassium thiocyanate, sodium acetate, sodium bromide, sodium malonate, sodium nitrate, sodium sulfate, and zinc acetate.

One of ordinary skill in the art will appreciate that other salts could be used as well, including, but are not limited to, NaCl and NaKPO₄.

One of two additional compounds, or no additional compound, can be included in each crystallization condition. These compounds are referred to as “additives”. Useful additives of the invention include, but are not limited to, glycerol and TMAO. The role of the salts and additives is to alter the surface and solution properties of the membrane proteins.

Some crystallization formulations of the invention are summarized in Table 3.

TABLE 3
Crystallization screen solution formulations.

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium chloride | 0.17 | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium chloride | 0.2 | | |
| 60 | MPEG 2000 | 31 | | | ammonium sulfate | 0.21 | | |
| 61 | MPEG 2000 | 29 | | | magnesium chloride | 0.17 | | |
| 62 | MPEG 2000 | 26 | | 0.1 | calcium chloride | 0.11 | | |
| 63 | MPEG 2000 | 27 | | | magnesium chloride | 0.16 | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium chloride | 0.24 | | |
| 67 | PEG 4000 | 16 | sodium citrate pH 4.5 | 0.1 | | | | |
| 68 | PEG 4000 | 14 | sodium citrate pH 4.5 | 0.1 | magnesium chloride | 0.21 | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium chloride | 0.2 | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium chloride | 0.21 | glycerol | 1.37% |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16 | | | | | | |

Some stock solutions encompassed by the invention that are useful for preparing the formulations of Table 2 or Table 3 are provided in Table 1 (see Examples).

It is difficult, in general, to produce quantities of membrane proteins sufficient for structural studies. Crystallization of membrane proteins for x-ray diffraction often requires crystallization trials in several different detergents. Efficient membrane protein crystallization screening can enable a realistic pursuit of a membrane protein structure. The present invention further provides a membrane protein crystallization screening kit which combines, for example, a solubility screen and a multiple-condition crystallization screen. In one aspect, the multiple-condition crystallization screen is a 96-condition crystallization screen. The solubility screen is used to ensure that a membrane protein, in a particular detergent, is at a concentration appropriate for use in the crystallization screen. The crystallization screen is a sparse matrix screen based on past successes in membrane protein crystallization. The membrane protein crystallization screening kit significantly outperformed commercially available membrane protein crystallization screens in the case of an alpha-helical membrane protein, AqpZ, and performed competitively in the case of a beta-barrel membrane protein, BtuB.

The present invention comprises the novel use of matching crystallization and solubility screens and multiple solutions. First, the crystallization screen is matched with a solubility screen which ensures that the protein is at a concentration which renders the crystallization screen effective. Second, the ninety-six formulation solutions of the membrane protein crystallization screen are novel and provide a quick and thorough set of formulations to ensure that crystallization occurs.

A commonly encountered problem in macromolecular crystallization experiments is failure to achieve supersaturated conditions in a sufficient number of crystallization conditions. This problem occurs either when the macromolecular concentration is too low or too high. Because the detergents used to maintain the solubility of purified membrane proteins can have a significant impact on the solution properties of the proteins, the solubility screen provides a rapid check of the appropriateness the protein concentration prior to crystallization screening in each detergent.

The crystallization screen is a sparse matrix sampling of PEG and MPEG molecular weight and concentration, and salts and buffers. The range of concentrations of the precipitants is broad to ensure that the screen is useful for membrane proteins with a wide range of solution properties. Additionally, the crystallization screen is detergent independent to accommodate a wide variety of detergents. Specifically, the precipitant and salt concentrations were selected such that excessive phase separation will not occur with most detergent containing solutions, when the detergent concentrations are near those commonly used to maintain the solubility of membrane proteins (i.e., several times the critical micelle concentration, CMC). However, the precipitant concentrations are high enough to result in membrane protein supersaturation, because, the solubility screen is used to determine the protein concentration necessary to ensure this state in a majority of the crystallization screen conditions.

The present invention comprises a solubility prescreen, which is matched to the crystallization screen, and crystallization conditions based on previously productive membrane protein crystallization conditions. The components of the crystallization screen solutions and the concentrations of the precipitating agents in the present invention efficiently sample a crystallization space defined by the previously productive membrane protein crystallization conditions, without the high redundancy of many existing membrane protein crystallization screens.

The potential impact of the present invention is more efficient membrane protein crystallization screening. There is also the potential for a higher success rate for membrane protein crystallization, based on the small number of membrane proteins tested with the present invention. Successful crystallization of membrane proteins has a very high scientific and potentially very high commercial impact, due to the biological, biomedical, and pharmaceutical importance of these macromolecules. The present invention is also well suited to the crystallization of membrane protein/soluble protein complexes, due to the similar solution conditions used to maintain the solubility of membrane proteins and membrane protein/soluble protein complexes. Structural biology of membrane protein complexes is a frontier area of science with high scientific and biomedical significance.

One of ordinary skill in the art will appreciate that the specific combination of prescreening solubility formulations disclosed herein could be used with a different set of crystallization formulations than described herein.

One of ordinary skill in the art will appreciate that the specific combination of crystallization formulations disclosed herein can be used with a different set of prescreening solubility formulations than described herein.

In one embodiment of the invention, the stock solutions of Table 1 (see Examples) are used to prepare the solutions of Table 2 and Table 3. In one aspect, a protein is tested for solubility using at least on formulation of the set of formulations of Table 2 (solubility formulations 1-24) and then screened using at least one of the ninety-six crystallization formulations of Table 3 (crystallization screen formulations 1-96).

The present invention further encompasses the use of multiwell plates and robotic apparatuses for performing the methods of the invention and for using the kits of the invention.

The present invention further provides a membrane protein solubility screening kit. In one aspect, the kit may also include the compositions of a membrane protein crystallization screening kit.

The present invention also provides a membrane protein crystallization screening kit. In one aspect, the kit may also include the compositions of a membrane protein solubility screening kit.

The formulations for solubility screening and for crystallization can be found in Tables 2 and 3. Kits may comprise various containers for the formulations, including, but not limited to, vials, tubes, and multiwell plates. Kits may comprise multiple samples of each formulation. Kits may further comprise standard protein samples.

### BRIEF DESCRIPTION OF THE DRAWINGS

FIG. 1 is a graphic representation of percentage of conditions (“Success Rate”—ordinate) in commercially available membrane protein crystallization screens and in the present invention that yielded crystals of two exemplary membrane proteins. Asterisks indicate that AqpZ did not crystallize in MemFacHT and that BtuB did not crystallize in Optimix-5. Black bars indicate BtuB; Gray bars indicate AqpZ.

### DETAILED DESCRIPTION OF THE INVENTION

### Abbreviations and Acronyms

Aquaporin Z—AqpZ

critical micelle concentration—CMC

dithiothreitol—DTT

free interface diffusion—FID

immobilized metal affinity chromatography—IMAC

molecular weight cutoff—MWCO

n-octyl-β-D-glucoside—OG

nuclear magnetic resonance spectroscopy—NMR

polyethylene glycol—PEG

polyethylene glycol monomethylether—MPEG

protein detergent complex—PDC

tris(2-carboxyethyl) phosphine hydrochloride—TCEP

### DEFINITIONS

In describing and claiming the invention, the following terminology will be used in accordance with the definitions set forth below. Unless defined otherwise, all technical and scientific terms used herein have the commonly understood meaning by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

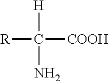

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

A “compound,” as used herein, refers to any type of substance or agent that is commonly considered a drug, or a candidate for use as a drug, as well as combinations and mixtures of the above. When referring to a compound of the invention, and unless otherwise specified, the term “compound” is intended to encompass not only the specified molecular entity but also its pharmaceutically acceptable, pharmacologically active analogs, including, but not limited to, salts, polymorphs, esters, amides, prodrugs, adducts, conjugates, active metabolites, and the like, where such modifications to the molecular entity are appropriate.

By the phrase “contacting a sample of a protein with at least one formulation” means that if more than one formulation is tested that the sample of the protein is either tested as an aliquot from the sample or that separate samples of the protein are prepared and used.

The use of the word “detect” and its grammatical variants is meant to refer to measurement of the species without quantification. The terms “detect” and “identify” are used interchangeably herein.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

A “formulation sample” or “sample of a formulation” means an aliquot of the designated formulation. When referenced in a kit, the aliquot is enough to perform at least one experiment for a protein sample. In one aspect, the formulation sample may be in a quantity such that multiple experiments can be performed. A kit may also contain multiple samples of each formulation.

As used herein, an “instructional material” includes a publication, a recording, a diagram, or any other medium of expression which can be used to communicate the usefulness of the formulations and methods of the invention in the kit. The instructional material of the kit of the invention may, for example, be affixed to a container which contains the identified formulations of invention or be shipped together with a container which contains the identified compound. Alternatively, the instructional material may be shipped separately from the container with the intention that the instructional material and the formulations be used cooperatively by the recipient.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes. The term “standard,” as used herein, refers to something used for comparison.

For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured.

### EMBODIMENTS

In one embodiment, the present invention comprises a 96-solution crystallization screen and a 24-condition solubility screen. In one aspect, the compositions and methods of the invention are useful for the crystallization of purified membrane proteins. However, the utility of the invention is not limited to crystallization of membrane proteins. The crystallization screen and solubility screen may be useful in the crystallization of other classes of macromolecules, including soluble proteins, nucleic acids, and complexes comprised of any combination of macromolecules.

In one embodiment, the present invention provides a method for identifying a formulation useful for determining the solubility of a protein, comprising contacting samples of a protein in at least one solubility formulation of the invention.

In one embodiment, the present invention provides a method for identifying a formulation useful for crystallizing a protein.

In one embodiment, the present invention provides a method of prescreening the solubility of a protein to better determine the concentration of protein to test in the crystallization formulation assay.

In one embodiment, the present invention provides a method of crystallizing a protein, comprising contacting a sample of protein with the crystallization formulation which has been predetermined to crystallize the protein, thereby crystallizing the protein.

Useful solutions of the invention include, but are not limited to, those described in Tables 1, 2, and 3. The solutions of the crystallization screen, Table 3, and the solubility screen, Table 2, are prepared by diluting the stock solutions, Table 1, in water to obtain the specified concentrations of each of the components. The solutions of the crystallization screen and the solubility screen can be prepared by a fluid handling robot or prepared manually using pipettes or by any other means that achieves the correct final concentrations of the components of the solutions of the screens. One of ordinary skill in the art will understand that, based on the methods and compositions disclosed herein, that the conditions and solutions can be modified and that the number of conditions and solutions used can be modified to study proteins of interest.

A description of a recommended implementation of the invention follows. Use of the invention is not limited to these applications. The crystallization screen can be used with any crystallization technique, including, but not limited to, vapor diffusion crystallization, microbatch crystallization, free interface diffusion (FID) crystallization, and microfluidic crystallization. The solubility screen can be used as described or in any way that aids in determining the optimal concentration of the object of crystallization. The solubility screen may also be used as a crystallization screen and has produced crystals of membrane proteins in vapor diffusion crystallization experiments. Therefore, when the solubility screen is used as described to determine the appropriate concentration of the object of crystallization, the experiment should be saved and examined regularly for crystals, as with a conventional crystallization screen.

In one embodiment of the invention, a membrane protein can be purified to the highest degree possible in the presence of one of more detergents that maintain the monodispersity of a membrane protein. A membrane protein can be concentrated to the highest degree that allows maintenance of the monodispersity of the protein. In one aspect, a membrane protein at a maximal concentration and half the maximal concentration can be used in the solubility screen. Specifically, a membrane protein can be mixed, typically at a 1:1 volumetric ratio, with each condition of the solubility screen in vapor diffusion crystallization experiments. One of ordinary skill in the art will appreciate that any common and useful vapor diffusion crystallization plate may be used to practice the invention. The experiments can be inspected after a period of thirty minutes for the presence and absence of membrane protein precipitate. In one aspect, twenty of the solubility screen solutions are present in pairs containing low or high concentrations of each of the precipitants (PEGs and MPEGs).

In one useful aspect, when a membrane protein concentration is appropriate for use in the crystallization screen, the screening conditions with low precipitant concentrations will be clear and the conditions with high precipitant concentrations will contain protein precipitate. If neither of the membrane protein samples (maximal and half maximal concentrations) exhibits the described behavior, additional testing with alternate protein concentrations is recommended. For example, if the majority of the conditions containing high precipitant concentrations are clear, even at the higher protein concentration, then the protein should be concentrated further and the screening repeated. Conversely, if the majority of the conditions containing low precipitant concentrations contain protein precipitate, even at the lower protein concentration, then lower concentration samples of the protein should be prepared and the screening should be repeated. The protein concentration that yields results that most closely follow the described trend is the concentration which should be used in the crystallization screen.

The solubility screen also includes two pairs of conditions which contain intermediate concentrations of precipitants and either acidic (pH 4.5) or basic (pH 8.5) pH. These conditions are intended to aid in the characterization of a membrane protein and in the interpretation of the crystallization screen experimental results. Specifically, these conditions are intended to reveal the possible presence of a sensitive dependence of the protein solubility on pH. Because pH may be an important variable in crystallization, a membrane protein should be minimally buffered to allow alteration of the solution pH by the buffers in the crystallization solutions.

One embodiment of the crystallization screen comprises mixing a solution containing a purified membrane protein, at the optimal concentration, with each of the solutions of the crystallization screen in a 1:1 volumetric ratio in vapor diffusion experiments. In one aspect, volumes of the crystallization drops (i.e., each mixture of the protein and a screen solution) are from about 100 nL to about 1.0 μL, depending on the method used to prepare the experiment. The recommended volume of the reservoirs (i.e., the corresponding solution of the screen which is present in excess in a sealed chamber with the drop in the vapor diffusion experiment) is about 100 to about 1,000 times the initial crystallization drop volume. The volume of the drop will be dictated in part by the available fluid handling technology. In the presently recommended implementation, the smallest volume drops that can be dispensed accurately and imaged optically can be used in both the solubility screen and crystallization screen. The primary goal of the solubility screen is to determine the appropriateness of the concentration of the membrane protein. Therefore, the only requirement for the volume of the experiment is the ability to inspect each experiment for the presence of protein precipitate. Similarly, the primary goal of the crystallization screen is to determine conditions that yield initial crystals of the membrane protein, and the only requirement for the volume of these experiments is the ability to inspect each experiment for the presence of crystals. It is assumed that some adjustment of the formulations of the conditions that yield crystals (optimization) will be required in most cases to obtain crystals suitable for x-ray diffraction experiments. Therefore, the ability to harvest crystals directly from the crystallization screen is not essential, and use of small volume crystallization experiments can substantially reduce the quantity of membrane protein required for the solubility screen and crystallization screen. For example, for an initial drop volume of 200 nL (100 nL protein) at a membrane protein concentration of 10 mg/mL, the membrane protein required for the crystallization screen is approximately 100 μg.

Descriptions of the application of the invention to two membrane proteins previously crystallized are included as examples of a recommended implementation of the invention. Also included are the results of crystallization experiments with the same two membrane proteins using currently commercially available membrane protein crystallization screens. These descriptions are provided only as examples of the implementation of the invention and are not intended to limit the application of the invention to the described methods or proteins.

One of ordinary skill in the art will appreciate that the present invention can be automated using the methods described herein as well as with other manual, robotic or automated apparatuses, such as those described in U.S. Pat. Nos. 6,267,935, 6,599,441, 6,916,455, 7,276,216, and 7,300,520. Aspects of the invention can also be performed manually, in whole or in part in conjunction with an automated system.

Crystallization using the methods of the invention can be done in more than one type of plate or device. For example, crystallization can be performed using multi-well plates or tubes.

In one embodiment, the invention provides compositions and methods useful for proteins. In one aspect, the protein is a membrane protein. In one aspect, the protein is associated with a disease, disorder, or condition.

The present invention further allows for variation of the described formulations of the invention while maintaining the activity described herein. For example, the concentrations of the reagents can be modified, the pH of the formulation solutions can be modified, the salts can be varied, and the additives can be varied.

### EXAMPLES

The solutions of the solubility screen and crystallization screen were prepared from stock solutions and Milli-Q (Millipore, Bellerica, Mass.) water using a Multiprobe HT fluid handling robot (PerkinElmer, Waltham, Mass.). 1.5 mL of each solution was prepared in a ninety-six well polypropylene plate (Fisher Scientific, Waltham, Mass.). In the case of the solubility screen, the screen was dispensed in quadruplicate, with each instance of the screen occupying two twelve well rows.

The E. coli water channel, Aquaporin Z (AqpZ), is an alpha helical plasma membrane protein. The protomer possesses six transmembrane alpha helices and two helices that span approximately half of the membrane. AqpZ was expressed and purified as described previously [4] with the following modifications. Cobalt resin was used for immobilized metal affinity chromatography (IMAC) purification. The polyhistidine tag was removed using an immobilized trypsin column. Subtractive IMAC and gel filtration purification were used to purify the protein after polyhistidine tag removal. Following purification, the protein was concentrated in a 50 kDa molecular weight cutoff (MWCO) centrifugal concentrator (Millipore, Bellerica, Mass.). AqpZ at concentrations of 8 mg/mL and 4 mg/mL in a solution containing 20 mM Tris pH 7.4, 100 mM NaCl, 10% glycerol, 2 mM dithiothreitol (DTT), and 40 mM n-octyl-β-D-glucoside (OG, Anatrace, Maumee, Ohio).

The E. coli cobalamin transporter, BtuB, is a 22-stranded beta barrel outer membrane protein [5]. BtuB was expressed and purified as described previously [6]. Following purification the protein was concentrated in a 50 kDa centrifugal concentrator to 10 mg/mL and then dialyzed against 20 mM Tris pH 8.0, 0.5 mM tris(2-carboxyethyl) phosphine hydrochloride (TCEP, Invitrogen, Carlsbad, Calif.), and 0.6% tetraethylene glycol monooctylether (C₈E₄, Anatrace). A sample of BtuB was diluted to 5 mg/mL with dialysis buffer for use in the solubility screen. Prior to use in the solubility screen CaCl₂ was added to the protein to a final concentration of 0.5 mM from a 50 mM CaCl₂ stock.

Experimental setup was identical for AqpZ and BtuB. The low and high concentration protein samples were mixed with the solutions of the solubility screen. The solubility screen was prepared as a vapor diffusion experiment using a Mosquito crystallization robot (TTP Labtech, Cambridge, Mass.) and a 96-well Innovaplate SD-2 crystallization plate (Hampton Research, Aliso Viejo, Calif.). The crystallization plate accommodates two sitting drops per reservoir well, which enables testing the two concentrations of protein within a single vapor diffusion chamber. In the case of the solubility screen, only two of the twelve-well rows of the plate were used. 90 μL of each solution was transferred from the deep well plate containing the screen solutions to the reservoir wells of the crystallization plate using a multichannel pipette. Protein for each drop well of the crystallization plate was supplied by an eight well plastic strip with a 2 μL capacity per well (TTP Labtech). Each well of each strip supplied protein to one twelve-well row of the crystallization plate. 1.7 μL of protein was dispensed into each well of the protein strips.

The crystallization plate was then loaded onto the crystallization robot. The crystallization robot dispensed 100 nL of the low concentration protein into each of the top drop wells, then, dispensed 100 nL of reservoir solution into each of those wells. Then, the same was done for the high concentration protein sample in the lower drop wells. The plate was sealed with Crystal Clear tape (Manco, Avon, Ohio) immediately after the drops were dispensed by the robot.

After a 30-minute incubation at 22° C., the drops of the solubility experiments were inspected with an optical microscope. The results from the solubility screen indicated that the lower concentrations of both AqpZ and BtuB were best for use in the crystallization screen. AqpZ and BtuB at the higher concentrations formed amorphous precipitate in the majority of the solubility screen conditions, including the low precipitant concentration conditions. AqpZ and BtuB at the lower concentrations (4 mg/mL and 5 mg/mL, respectively) formed amorphous precipitate in the majority of the high precipitant conditions, but did not precipitate in the low precipitant concentration conditions of the solubility screen.

The crystallization screen of the present invention and the existing commercially available membrane protein crystallization screens were prepared as vapor diffusion crystallization experiments exactly as described above for the solubility screen. The commercially available screens were tested for comparison with the present invention. The screens tested were MemFac™ HT (Hampton Research, Aliso Viejo, Calif.), OptiMix-5 Membrane (Fluidigm, South San Francisco, Calif.), JBScreen Membrane HTS (Jena Bioscience, Jena, Germany), MbClass Suite and MbClass II Suite (QIAGEN, Valencia, Calif.), MemGold™ HT-96 (Molecular Dimension USA, Apopka, Fla.). All of these screens are 96-condition crystallization screens, with the exception of JBScreen Membrane HTS which is a 64-condition screen.

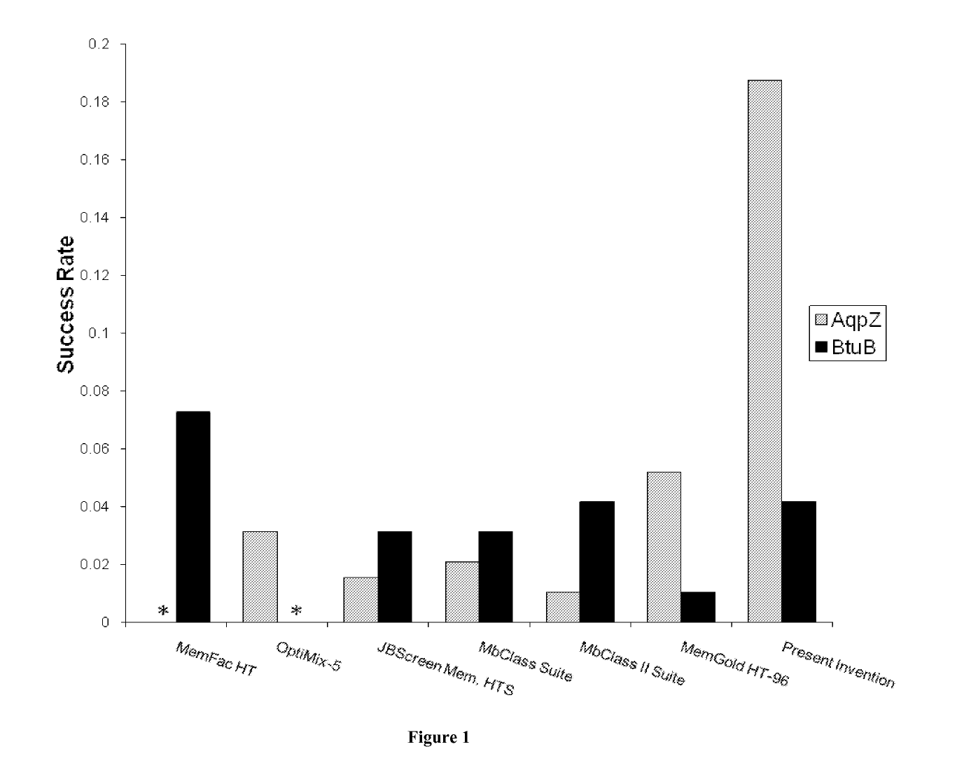

The crystallization experiments were imaged with a CrystalPro HT automated imager (TriTek, Sumerduck, Va.). Drop images were collected immediately after setup, after 24 hours and every two days for two weeks. A drop was considered to contain crystals of the membrane protein when the following conditions were met: there were no crystals in the drop immediately after setup, crystals with clearly defined edges were visible, and there was no evidence suggesting the solution favored formation of small molecule crystals. The results of the crystallization experiments are summarized in FIG. 1.

TABLE 1
Stock Solutions

| | |
|---|---|
| PEG 400 | 100.0% |
| PEG 1000 | 50.0% |
| PEG 4000 | 50.0% |
| MPEG 550 | 100.0% |
| MPEG 2000 | 50.0% |
| MPEG 5000 | 50.0% |
| lithium sulfate | 2.0M |
| lithium acetate | 2.0M |
| lithium chloride | 2.0M |
| lithium nitrate | 2.0M |
| sodium acetate | 2.0M |
| sodium bromide | 2.0M |
| sodium sulfate | 1.0M |
| sodium nitrate | 2.0M |
| potassium phosphate dibasic | 2.0M |
| potassium chloride | 2.0M |
| potassium thiocyanate | 2.0M |
| ammonium sulfate | 2.0M |
| ammonium nitrate | 2.0M |
| magnesium sulfate | 2.0M |
| magnesium acetate | 2.0M |
| magnesium chloride | 2.0M |
| calcium chloride | 2.0M |
| zinc acetate | 1.5M |
| sodium malonate | 2.0M |
| glycerol | 35.0% |
| trimethylamine oxide, TMAO | 3.0M |
| sodium citrate pH 4.5 | 1.0M |
| sodium acetate pH 5.5 | 2.0M |
| ADA pH 6.5 | 0.5M |
| HEPES pH 7.5 | 1.0M |
| Tris-HCl pH 8.5 | 1.0M |
| CAPSO pH 9.5 | 0.5M |

TABLE 2
Solubility screen solution formulations.

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1 |

TABLE 3
Crystallization screen solution formulations.

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium chloride | 0.17 | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium chloride | 0.2 | | |
| 60 | MPEG 2000 | 31 | | | ammonium sulfate | 0.21 | | |
| 61 | MPEG 2000 | 29 | | | magnesium chloride | 0.17 | | |
| 62 | MPEG 2000 | 26 | | 0.1 | calcium chloride | 0.11 | | |
| 63 | MPEG 2000 | 27 | | | magnesium chloride | 0.16 | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium chloride | 0.24 | | |
| 67 | PEG 4000 | 16 | sodium citrate pH 4.5 | 0.1 | | | | |
| 68 | PEG 4000 | 14 | sodium citrate pH 4.5 | 0.1 | magnesium chloride | 0.21 | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium chloride | 0.2 | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium chloride | 0.21 | glycerol | 1.37% |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16 | | | | | | |

One of ordinary skill in the art will appreciate that other methods useful for the practice of the present of the invention may not necessarily be described herein and that methods known in the art may be applicable to the practice of the present invention. Other methods useful in the practice of the invention include, but are not limited to, those found in U.S. Pat. Nos. 6,267,935, 6,599,441, 6,916,455, 7,276,216, and 7,300,520.

The disclosures of each and every patent, patent application, and publication cited herein are hereby incorporated by reference herein in their entirety. One of skill in the art will appreciate that the superiority of the compositions and methods of the invention relative to the compositions and methods of the prior art are unrelated to the physiological accuracy of the theory explaining the superior results.

Headings are included herein for reference and to aid in locating certain sections. These headings are not intended to limit the scope of the concepts described therein under, and these concepts may have applicability in other sections throughout the entire specification.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

### BIBLIOGRAPHY

The references as cited throughout this document and below are hereby incorporated by reference herein in their entirety.
1. Weber, P. C. (1991) Physical principles of protein crystallization. Adv Protein Chem 41, 1-36
2. Ahram, M., Litou, Z. I., Fang, R. and Al Tawallbeh, G. (2006) Estimation of membrane proteins in the human proteome. In Silico Biology 6
3. Arakawa, T. and Timasheff, S. N. (1985) Theory of protein solubility. Methods Enzymol 114, 49-77
4. Savage, D. F., Egea, P. F., Robles Colmenares, Y., O'Connell, J. D., 3rd and Stroud, R. M. (2003) Architecture and selectivity in aquaporins: 2.5 Å X ray structure of aquaporin Z. PLoS Biol 1, E72
5. Chimento, D. P., Mohanty, A. K., Kadner, R. J. and Wiener, M. C. (2003) Substrate induced transmembrane signaling in the cobalamin transporter BtuB. Nat Struct Biol 10, 394-401
6. Chimento, D. P., Mohanty, A. K., Kadner, R. J. and Wiener, M. C. (2003) Crystallization and initial X ray diffraction of BtuB, the integral membrane cobalamin transporter of Escherichia coli. Acta Crystallogr D Biol Crystallogr 59, 509-511

What is claimed is:

1. A set of protein solubility screening formulations for determining the solubility of a protein, said set comprising the twenty-four formulations of the following table:

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1 |

2. The set of protein solubility screening formulations of claim 1, wherein said protein is a membrane protein.

3. A kit for prescreening the solubility of a protein, said kit comprising at least one sample of at least one protein solubility screening formulation of claim 1, and an instructional material for the use thereof.

4. The kit of claim 3, wherein said kit comprises at least one sample of all twenty-four solubility screening formulations.

5. A set of protein crystallization screening formulations for identifying a formulation for crystallizing a protein, said set comprising the ninety-six formulations of the following table:

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium | 0.17 | | |
| | | | | | chloride | | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium | 0.2 | | |
| | | | | | chloride | | | |
| 60 | MPEG 2000 | 31 | | | ammonium sulfate | 0.21 | | |
| 61 | MPEG 2000 | 29 | | | magnesium | 0.17 | | |
| | | | | | chloride | | | |
| 62 | MPEG 2000 | 26 | | 0.1 | calcium chloride | 0.11 | | |
| 63 | MPEG 2000 | 27 | | | magnesium | 0.16 | | |
| | | | | | chloride | | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium | 0.24 | | |
| | | | | | chloride | | | |
| 67 | PEG 4000 | 16 | sodium citrate pH 4.5 | 0.1 | | | | |
| 68 | PEG 4000 | 14 | sodium citrate pH 4.5 | 0.1 | magnesium | 0.21 | | |
| | | | | | chloride | | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium | 0.2 | | |
| | | | | | chloride | | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium | 0.21 | glycerol | 1.37% |
| | | | | | chloride | | | |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16 | | | | | | |

6. The set of protein solubility screening formulations of claim 5, wherein said protein is a membrane protein.

7. A kit for screening formulations to identify a formulation for crystallizing a protein, said kit comprising at least one sample of at least one formulation of claim 5, and an instructional material for the use thereof.

8. A method for screening formulations for determining the solubility of a protein and for identifying a formulation for crystallizing a protein, said method comprising:
contacting a sample of said protein with at least one formulation for determining the solubility of a protein;
determining the solubility of the protein in the formulation;
contacting a sample of said protein with at least one formulation for crystallizing a protein,
determining whether said protein crystallizes,
thereby screening formulations for determining the solubility of a protein and for identifying a formulation for crystallizing a protein.

9. The method of claim 8, wherein the amount of said protein contacted with said at least one crystallization formulation is based on the solubility of said protein in said at least one solubility formulation.

10. The method of claim 8, wherein at least two concentrations of said protein are tested in said at least one solubility screening formulation.

11. The method of claim 8, wherein at least two solubility formulations are tested.

12. The method of claim 8, wherein twenty-four solubility formulations are tested.

13. The method of claim 8, wherein said at least one solubility formulation is selected from the twenty-four formulations of the table:

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | ithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1 |

14. The method of claim 8, wherein at least two crystallization formulations are tested.

15. The method of claim 8, wherein ninety-six crystallization formulations are tested.

16. The method of claim 8, wherein said at least one crystallization formulation is selected from the formulations of the following table:

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium | 0.17 | | |
| | | | | | chloride | | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium | 0.2 | | |
| | | | | | chloride | | | |
| 60 | MPEG 2000 | 31 | | | ammonium sulfate | 0.21 | | |
| 61 | MPEG 2000 | 29 | | | magnesium | 0.17 | | |
| | | | | | chloride | | | |
| 62 | MPEG 2000 | 26 | | 0.1 | calcium chloride | 0.11 | | |
| 63 | MPEG 2000 | 27 | | | magnesium | 0.16 | | |
| | | | | | chloride | | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium | 0.24 | | |
| | | | | | chloride | | | |
| 67 | PEG 4000 | 16 | sodium citrate pH 4.5 | 0.1 | | | | |
| 68 | PEG 4000 | 14 | sodium citrate pH 4.5 | 0.1 | magnesium | 0.21 | | |
| | | | | | chloride | | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium | 0.2 | | |
| | | | | | chloride | | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium | 0.21 | glycerol | 1.37% |
| | | | | | chloride | | | |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16 | | | | | | |

17. A kit for screening formulations for determining the solubility of a protein and for identifying a formulation for crystallizing a protein, said kit comprising:
at least one solubility formulation sample selected from the twenty-four formulations of the table:

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1; |

at least one crystallization formulation sample selected from the formulations of the following table:

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium | 0.17 | | |
| | | | | | chloride | | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium | 0.2 | | |
| | | | | | chloride | | | |
| 60 | MPEG 2000 | 31 | | | ammonium sulfate | 0.21 | | |
| 61 | MPEG 2000 | 29 | | | magnesium | 0.17 | | |
| | | | | | chloride | | | |
| 62 | MPEG 2000 | 26 | | 0.1 | calcium chloride | 0.11 | | |
| 63 | MPEG 2000 | 27 | | | magnesium | 0.16 | | |
| | | | | | chloride | | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium | 0.24 | | |
| | | | | | chloride | | | |
| 67 | PEG 4000 | 16 | sodium citrate pH 4.5 | 0.1 | | | | |
| 68 | PEG 4000 | 14 | sodium citrate pH 4.5 | 0.1 | magnesium | 0.21 | | |
| | | | | | chloride | | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium | 0.2 | | |
| | | | | | chloride | | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium | 0.21 | glycerol | 1.37% |
| | | | | | chloride | | | |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16; | | | | | | |

and an instructional material for the use thereof.

18. The kit of claim 17, wherein said kit comprises at least one sample for all twenty-four solubility formulations.

19. The kit of claim 17, wherein said kit comprises at least one sample for all ninety-six crystallization formulations.

20. The kit of claim 17, wherein said kit comprises at least one sample for all twenty-four solubility formulations and at least one sample for all ninety-six crystallization formulations.

21. A method for crystallizing a protein, said method comprising:
contacting a sample of said protein with at least one screening formulation for determining the solubility of a protein and determining the solubility of said protein;
preparing a solution of said protein based on the solubility that was determined; and
contacting a sample of said solution with at least one crystallization formulation;
thereby crystallizing said protein.

22. The method of claim 21, wherein said at least one screening formulation for determining the solubility of a protein is selected from the twenty-four formulations of the table:

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1. |

23. The method of claim 22, wherein all twenty-four solubility screening formulations are used.

24. The method of claim 21, wherein said at least one crystallization formulation is selected from the ninety-six formulations of the table:

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium | 0.17 | | |
| | | | | | chloride | | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium | 0.2 | | |
| | | | | | chloride | | | |
| 60 | MPEG 2000 | 31 | | | ammonium sulfate | 0.21 | | |
| 61 | MPEG 2000 | 29 | | | magnesium | 0.17 | | |
| | | | | | chloride | | | |
| 62 | MPEG 2000 | 26 | | 0.1 | calcium chloride | 0.11 | | |
| 63 | MPEG 2000 | 27 | | | magnesium | 0.16 | | |
| | | | | | chloride | | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium | 0.24 | | |
| | | | | | chloride | | | |
| 67 | PEG 4000 | 16 | sodium citrate pH 4.5 | 0.1 | | | | |
| 68 | PEG 4000 | 14 | sodium citrate pH 4.5 | 0.1 | magnesium | 0.21 | | |
| | | | | | chloride | | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium | 0.2 | | |
| | | | | | chloride | | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium | 0.21 | glycerol | 1.37% |
| | | | | | chloride | | | |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16. | | | | | | |

25. The method of claim 24, wherein all ninety-six crystallization formulations are used.

26. A protein solubility screening formulation selected from the formulations of the table:

| | | PEG | | Buffer | | Salt |
|---|---|---|---|---|---|---|
| # | PEG | (%) | Buffer | (M) | Salt | (M) |
| 1 | PEG 400 | 50 | | | | |
| 2 | PEG 400 | 25 | | | | |
| 3 | MPEG 550 | 40 | | | | |
| 4 | MPEG 550 | 20 | | | | |
| 5 | MPEG 2000 | 35 | | | lithium sulfate | 0.1 |
| 6 | MPEG 2000 | 17 | | | lithium sulfate | 0.1 |
| 7 | PEG 1000 | 36 | | | | |
| 8 | PEG 1000 | 18 | | | | |
| 9 | MPEG 2000 | 35 | | | | |
| 10 | MPEG 2000 | 17 | | | | |
| 11 | MPEG 2000 | 35 | | | potassium | 0.1 |
| | | | | | thiocyanate | |
| 12 | MPEG 2000 | 17 | | | potassium | 0.1 |
| | | | | | thiocyanate | |
| 13 | PEG 4000 | 25 | | | | |
| 14 | PEG 4000 | 12 | | | | |
| 15 | MPEG 5000 | 22 | | | | |
| 16 | MPEG 5000 | 11 | | | | |
| 17 | MPEG 2000 | 35 | | | magnesium | 0.1 |
| | | | | | acetate | |
| 18 | MPEG 2000 | 17 | | | magnesium | 0.1 |
| | | | | | acetate | |
| 19 | PEG 1000 | 30 | sodium | 0.1 | | |
| | | | citrate | | | |
| | | | pH 4.5 | | | |
| 20 | PEG 1000 | 30 | Tris-HCl | 0.1 | | |
| | | | pH 8.5 | | | |
| 21 | MPEG 2000 | 25 | sodium | 0.1 | | |
| | | | citrate | | | |
| | | | pH 4.5 | | | |
| 22 | MPEG 2000 | 25 | Tris-HCl | 0.1 | | |
| | | | pH 8.5 | | | |
| 23 | MPEG 2000 | 35 | | | ammonium nitrate | 0.1 |
| 24 | MPEG 2000 | 17 | | | ammonium nitrate | 0.1. |

19. A protein crystallization screening formulation selected from the formulations of the table:

| | | PEG | | Buffer | | Salt | | Additive |
|---|---|---|---|---|---|---|---|---|
| # | PEG | (%) | Buffer | (M) | Salt | (M) | Additive | (% or M) |
| 1 | PEG 400 | 48 | sodium citrate pH 4.5 | 0.1 | sodium acetate | 0.21 | glycerol | 1.12% |
| 2 | PEG 400 | 32 | sodium acetate pH 5.5 | 0.1 | potassium chloride | 0.12 | | |
| 3 | PEG 400 | 46 | sodium acetate pH 5.5 | 0.1 | sodium malonate | 0.23 | | |
| 4 | PEG 400 | 42 | ADA pH 6.5 | 0.1 | magnesium acetate | 0.29 | | |
| 5 | PEG 400 | 45 | HEPES pH 7.5 | 0.1 | sodium acetate | 0.18 | TMAO | 0.45M |
| 6 | PEG 400 | 28 | Tris HCl pH 8.5 | 0.1 | lithium nitrate | 0.29 | | |
| 7 | PEG 400 | 36 | CAPSO pH 9.5 | | magnesium | 0.17 | | |
| | | | | | chloride | | | |
| 8 | PEG 400 | 36 | | | magnesium sulfate | 0.12 | glycerol | 3.67% |
| 9 | PEG 400 | 47 | | | ammonium sulfate | 0.17 | | |
| 10 | PEG 400 | 41 | | | sodium malonate | 0.22 | | |
| 11 | PEG 400 | 31 | | | sodium sulfate | 0.04 | | |
| 12 | PEG 400 | 32 | | | sodium sulfate | 0.24 | | |
| 13 | MPEG 550 | 30 | sodium citrate pH 4.5 | 0.1 | | | glycerol | 3.22% |
| 14 | MPEG 550 | 37 | sodium acetate pH 5.5 | 0.1 | | | | |
| 15 | MPEG 550 | 25 | sodium acetate pH 5.5 | 0.1 | calcium chloride | 0.2 | | |
| 16 | MPEG 550 | 33 | ADA pH 6.5 | 0.1 | magnesium sulfate | 0.17 | | |
| 17 | MPEG 550 | 38 | ADA pH 6.5 | 0.1 | sodium bromide | 0.14 | | |
| 18 | MPEG 550 | 39 | HEPES pH 7.5 | 0.1 | calcium chloride | 0.1 | | |
| 19 | MPEG 550 | 34 | Tris HCl pH 8.5 | 0.1 | ammonium nitrate | 0.22 | | |
| 20 | MPEG 550 | 23 | CAPSO pH 9.5 | 0.1 | lithium chloride | 0.06 | | |
| 21 | MPEG 550 | 29 | | | K phosphate dib | 0.29 | | |
| 22 | MPEG 550 | 23 | | | sodium bromide | 0.28 | | |
| 23 | MPEG 550 | 22 | | | | | | |
| 24 | MPEG 550 | 30 | | | calcium chloride | 0.2 | | |
| 25 | MPEG 550 | 32 | | | sodium malonate | 0.22 | | |
| 26 | MPEG 550 | 38 | | | magnesium acetate | 0.16 | | |
| 27 | MPEG 550 | 33 | | | sodium acetate | 0.25 | | |
| 28 | PEG 1000 | 25 | sodium citrate pH 4.5 | 0.1 | potassium chloride | 0.19 | | |
| 29 | PEG 1000 | 35 | sodium citrate pH 4.5 | 0.1 | ammonium nitrate | 0.19 | | |
| 30 | PEG 1000 | 27 | sodium acetate pH 5.5 | 0.1 | ammonium nitrate | 0.14 | | |
| 31 | PEG 1000 | 34 | ADA pH 6.5 | 0.1 | | | | |
| 32 | PEG 1000 | 20 | HEPES pH 7.5 | 0.1 | ammonium nitrate | 0.2 | | |
| 33 | PEG 1000 | 28 | HEPES pH 7.5 | 0.1 | sodium malonate | 0.1 | | |
| 34 | PEG 1000 | 22 | Tris HCl pH 8.5 | 0.1 | | | | |
| 35 | PEG 1000 | 33 | Tris HCl pH 8.5 | 0.1 | | | | |
| 36 | PEG 1000 | 35 | CAPSO pH 9.5 | 0.1 | potassium chloride | 0.18 | | |
| 37 | PEG 1000 | 28 | CAPSO pH 9.5 | 0.1 | lithium chloride | 0.29 | | |
| 38 | PEG 1000 | 19 | | | lithium nitrate | 0.24 | | |
| 39 | PEG 1000 | 33 | | | ammonium sulfate | 0.27 | | |
| 40 | PEG 1000 | 29 | | | sodium bromide | 0.21 | glycerol | 3.80% |
| 41 | PEG 1000 | 32 | | | | | glycerol | 2.35% |
| 42 | PEG 1000 | 35 | | | potassium chloride | 0.18 | glycerol | 4.16% |
| 43 | PEG 1000 | 36 | | | zinc acetate | 0.02 | | |
| 44 | | 0 | | | ammonium sulfate | 2 | | |
| 45 | MPEG 2000 | 34 | sodium citrate pH 4.5 | 0.1 | | | TMAO | 0.54M |
| 46 | MPEG 2000 | 28 | sodium citrate pH 4.5 | 0.1 | | | | |
| 47 | MPEG 2000 | 26 | sodium citrate pH 4.5 | 0.1 | zinc acetate | 0.02 | | |
| 48 | MPEG 2000 | 23 | sodium acetate pH 5.5 | 0.1 | sodium acetate | 0.1 | | |
| 49 | MPEG 2000 | 29 | sodium acetate pH 5.5 | 0.1 | sodium nitrate | 0.15 | | |
| 50 | MPEG 2000 | 28 | sodium acetate pH 5.5 | 0.1 | | | | |
| 51 | MPEG 2000 | 26 | ADA pH 6.5 | 0.1 | sodium sulfate | 0.13 | | |
| 52 | MPEG 2000 | 18 | ADA pH 6.5 | 0.1 | | | | |
| 53 | MPEG 2000 | 29 | HEPES pH 7.5 | 0.1 | lithium chloride | 0.29 | | |
| 54 | MPEG 2000 | 17 | Tris HCl pH 8.5 | 0.1 | lithium nitrate | 0.28 | | |
| 55 | MPEG 2000 | 34 | Tris HCl pH 8.5 | 0.1 | zinc acetate | 0.02 | | |
| 56 | MPEG 2000 | 29 | CAPSO pH 9.5 | 0.1 | | | | |
| 57 | MPEG 2000 | 17 | CAPSO pH 9.5 | 0.1 | lithium sulfate | 0.12 | | |
| 58 | MPEG 2000 | 18 | | | magnesium sulfate | 0.27 | | |
| 59 | MPEG 2000 | 22 | | | magnesium | 0.2 | | |
| | | | | | chloride | | | |
| 60 | MPEG 2000 | 31 | | | ammonium sulfate | 0.21 | | |
| 61 | MPEG 2000 | 29 | | | magnesium | 0.17 | | |
| | | | | | chloride | | | |
| 62 | MPEG 2000 | 26 | | 0.1 | calcium chloride | 0.11 | | |
| 63 | MPEG 2000 | 27 | | | magnesium | 0.16 | | |
| | | | | | chloride | | | |
| 64 | MPEG 2000 | 30 | | | sodium acetate | 0.23 | | |
| 65 | MPEG 2000 | 27 | | | sodium acetate | 0.13 | | |
| 66 | MPEG 2000 | 33 | | | magnesium | 0.24 | | |
| | | | | | chloride | | | |
| 67 | PEG 4000 | 16 | sodium citrate pH 4.5 | 0.1 | | | | |
| 68 | PEG 4000 | 14 | sodium citrate pH 4.5 | 0.1 | magnesium | 0.21 | | |
| | | | | | chloride | | | |
| 69 | PEG 4000 | 16 | sodium acetate pH 5.5 | 0.1 | ammonium nitrate | 0.12 | | |
| 70 | PEG 4000 | 20 | sodium acetate pH 5.5 | 0.1 | | | | |
| 71 | PEG 4000 | 16 | sodium acetate pH 5.5 | 0.1 | | | TMAO | 0.87M |
| 72 | PEG 4000 | 20 | sodium acetate pH 5.5 | 0.1 | magnesium | 0.2 | | |
| | | | | | chloride | | | |
| 73 | PEG 4000 | 13 | ADA pH 6.5 | 0.1 | sodium acetate | 0.29 | | |
| 74 | PEG 4000 | 19 | ADA pH 6.5 | 0.1 | lithium nitrate | 0.05 | | |
| 75 | PEG 4000 | 16 | ADA pH 6.5 | 0.1 | zinc acetate | 0.02 | | |
| 76 | PEG 4000 | 17 | HEPES pH 7.5 | 0.1 | ammonium nitrate | 0.27 | | |
| 77 | PEG 4000 | 13 | HEPES pH 7.5 | 0.1 | ammonium nitrate | 0.15 | TMAO | 0.42M |
| 78 | PEG 4000 | 13 | Tris HCl pH 8.5 | 0.1 | lithium sulfate | 0.11 | glycerol | 1.67% |
| 79 | PEG 4000 | 12 | Tris HCl pH 8.5 | 0.1 | lithium acetate | 0.24 | | |
| 80 | PEG 4000 | 21 | Tris HCl pH 8.5 | 0.1 | | | | |
| 81 | PEG 4000 | 18 | Tris HCl pH 8.5 | 0.1 | K phosphate dib | 0.27 | | |
| 82 | PEG 4000 | 14 | CAPSO pH 9.5 | 0.1 | sodium sulfate | 0.2 | | |
| 83 | PEG 4000 | 20 | CAPSO pH 9.5 | 0.1 | | | | |
| 84 | PEG 4000 | 23 | | | sodium bromide | 0.28 | | |
| 85 | PEG 4000 | 13 | | | magnesium | 0.21 | glycerol | 1.37% |
| | | | | | chloride | | | |
| 86 | PEG 4000 | 20 | | | potassium | 0.23 | glycerol | 1.11% |
| | | | | | thiocyanate | | | |
| 87 | MPEG 5000 | 18 | sodium citrate pH 4.5 | 0.1 | magnesium sulfate | 0.22 | | |
| 88 | MPEG 5000 | 17 | sodium acetate pH 5.5 | 0.1 | lithium nitrate | 0.25 | glycerol | 4.57% |
| 89 | MPEG 5000 | 18 | sodium acetate pH 5.5 | 0.1 | sodium sulfate | 0.22 | TMAO | 0.47M |
| 90 | MPEG 5000 | 20 | ADA pH 6.5 | 0.1 | lithium sulfate | 0.07 | | |
| 91 | MPEG 5000 | 12 | HEPES pH 7.5 | 0.1 | ammonium sulfate | 0.18 | | |
| 92 | MPEG 5000 | 20 | Tris HCl pH 8.5 | 0.1 | sodium sulfate | 0.2 | | |
| 93 | MPEG 5000 | 15 | CAPSO pH 9.5 | 0.1 | lithium nitrate | 0.15 | | |
| 94 | MPEG 5000 | 13 | | | calcium chloride | 0.05 | | |
| 95 | MPEG 5000 | 14 | | | zinc acetate | 0.02 | | |
| 96 | MPEG 5000 | 16. | | | | | | |
